(19) United States
(12) Patent Application Publication
(10) Pub. No.: US 2008/0004538 A1
(43) Pub. Date: Jan. 3, 2008

(54) MEDICAL TELEMETRY SYSTEM

(76) Inventor: Juha Virtanen, Helsinki (FI)

Correspondence Address: GENERAL ELECTRIC CO.;GLOBAL PATENT OPERATION, 187 Danbury Road, Suite 204, Wilton, CT, 06897-4122, US

(21) Appl. No.: 11/762,162

(22) Filed: Jun. 13, 2007

(30) Foreign Application Priority Data
Jun. 29, 2006 (EP) 06396011.6

(51) Int. Cl. A61B 5/0402

(52) U.S. Cl. 600/509

(57) ABSTRACT

A medical telemetry system and a method for signaling leads-off information in a medical telemetry system. The medical telemetry system according to an embodiment of the invention comprises a sensor unit comprising ECG electrodes connected to a transmitter unit, and a receiver unit connectable to ECG input connector on a patient monitor. The ECG signal and leads-off state of the electrodes are transmitted from the sensor unit to the receiver unit using radio-wave communication. Leads-off information detected by the sensor unit is transmitted to the patient monitor and signaled by feeding a predefined low-impedance DC-voltage to the ECG signal port.

### FIELD OF THE INVENTION

The field of the invention relates to a medical telemetry system and a method for signaling leads-off information in a medical telemetry system. The medical telemetry system according to the invention comprises a sensor unit comprising ECG electrodes connected to a transmitter unit, and a receiver unit connectable to ECG input connector on a patient monitor. The ECG signal and leads-off state of the electrodes are transmitted from the sensor unit to the receiver unit using radio-wave communication. According to an embodiment of the present invention, leads-off information to the patient monitor is signaled by feeding a predefined low-impedance DC-voltage to the ECG signal port.

### BACKGROUND OF THE INVENTION

Electrocardiography (ECG) measures the electrical activity of the heart. It depicts the rate and the regularity of heartbeat as well as the presence of cardiac diseases or damage, arrhythmias etc. ECG is measured by placing electrodes on the chest of the patient and measuring the bioelectrical potentials produced by the heart. Electrodes attached to the patient are connected by leads to an ECG monitor for further signal processing.

If there is a poor connection between the electrode and the patient's skin, the ECG signal will become distorted by noise. Detecting the poor electrode-to-skin connection or a lead detachment is important for the medical professionals to quickly locate the faulty connection and take appropriate steps to recover the measurement. Conventional leads-off detectors use impedance measurement in determining a bad electrode-to-skin connection.

FIG. 1 depicts a simplified prior art leads-off detecting circuit. The impedance of the electrodes is measured by continuously feeding a small (of the order of 10 nA) DC current through the ECG electrodes and the patient to ground through a reference electrode. The other input of the amplifier is thus connected to floating ground. If an electrode is loose or poorly connected, even such a small de current is adequate to create a considerable difference in the voltage measured from the electrode (the magnitude of the voltage may be a couple of volts). In case of leads-off, the resistance R1 in FIG. 1 forces the input of the amplifier A1 to rise to the predetermined DC-voltage of e.g. 2,5V or 5V. The voltage produced by the dc current is then compared with a reference voltage V_{ref} in comparator CO1 to determine if the impedance of the electrode is too high. When the output or the input of the second amplifier rises over the predetermined DC-voltage, it causes that the comparator switches its state and leads-off state is detected. Similarly, if the measuring electrodes are short-circuited, the input of the amplifier goes to zero volts and the comparator CO1 switches its state. Similar leads-off detection circuit is described in publication U.S. Pat. No. 4,577,639 (Simon, Mark I. et al).

Often, the monitored patient has to be moved from one location to another e.g. from the emergency rescue scene to the hospital or from one hospital unit to another. Detaching and reattaching the electrodes is time-consuming to the medical professionals and stressful to the patient, and therefore, highly undesirable. Free relative movement of the patient and the monitor is often desirable, but restricted by the measurement cable connecting the patient to the monitor. Furthermore, the typically thick and long cable between the patient and the monitor clutters easily and tends to be a nuisance for the nursing staff. In attempt to solve these problems, wireless ECGs have been developed. Publication EP 1551503 (Massicotte et al.) describes an example of a wireless ECG monitoring system. The signal acquired by electrodes attached to the patient is converted into a digital radio signal which is transmitted to a wireless receiver device. The receiver device can further be connected to a computer or a hand-held PC.

Patent publication U.S. Pat. No. 6,267,723 describes a medical telemetry system in which a sensor unit detects a biomedical signal which is converted into a radio signal by a transmitter. Transmission of medical signals via radio transmission is well known as telemetry. The transmitter described in the publication has an electrode detachment detection unit for detecting electrode detachment based on the output signal of the sensor unit. The biometric signal and the signal indicative of the electrode detachment are then transmitted and received by a receiver. Simplified electrical circuitry of a similar receiver is depicted in FIG. 2. The signal received from the transmitter is supplied to an amplifier A3 with a low input impedance, and further to an input of another device (the ECG monitor) through a switch SW1 and a connection unit CU1. If the electrode detachment signal is detected the switch is opened and the output impedance of the receiver's voltage output is thus changed. The switch simulates the real behavior of loose electrodes by disconnecting the measurement signal from the receiver unit and is thus an obvious and universal solution. When an ECG signal is input there is a very low output impedance, but in case of lead detachment the switch is opened and the output impedance is changed to very high. The publication also makes a difference when the radio wave transmission is interrupted versus when the electrodes are detached. The system described in the publication requires complicated control logic and, a large number of components requiring significantly board space is needed to implement electrode detachment circuitry. This is expensive and may lead to unreliable or false alarms.

Further problem in prior art systems is that A/D conversion of an ECG signal with a large offset voltage is not practical or possible to arrange without radically decreasing the resolution of the conversion. It is thus necessary to perform detecting of the leads-off state before digital transmission of the signal to the ECG monitor.

### SUMMARY OF THE INVENTION

The invention relates to a medical telemetry system and a method for signaling leads-off information in a medical telemetry system.

A purpose of the invention is to provide a simple and reliable method and system for signaling leads-off information in a medical telemetry system.

The medical telemetry system according to the invention comprises a patient monitor comprising an ECG input connector and a signal port, and a sensor unit in close proximity with the patient further comprising a transmitter unit and ECG electrodes to be attached on a patient and to be connected to the transmitter unit. The system comprises a receiver unit connected to an ECG input connector on the patient monitor, and configured to receive ECG signal and leads-off state information via wireless radio-wave communication from the transmitter unit, and to feed a reconstructed ECG signal to an input connector of the patient monitor. The leads-off information, detected by the sensor unit, is transmitted to the patient monitor and signaled by feeding a predefined DC-voltage to the ECG signal port by a signaling unit comprising at least a voltage source controlled by the leads-off state information.

An embodiment of the invention also relates to a method for signaling leads-off information in a medical telemetry system, the method comprising: arranging a sensor unit in proximity with the patient, the sensor unit comprising a transmitter unit and ECG electrodes to be attached on a patient and to be connected to the transmitter unit, receiving by a receiver unit connected to ECG input connector on a patient monitor comprising an ECG signal port, ECG signal and leads-off state information via wireless radio-wave communication from the transmitter unit, and feeding by said receiver unit a reconstructed ECG signal to an input connector of the patient monitor. The method further comprises the step of signaling the leads-off information to the ECG monitor by feeding a predefined DC-voltage to the ECG signal port.

The output impedance of the voltage source is low. Typically it is in the range between 1 and 100 ohms. In any case it should be less than 100E6 ohms.

In one embodiment of the invention the output impedance of the receiver unit is arranged to remain constant regardless of the leads-off information. The output impedance of the amplifier unit in the receiver does thus not change when changing over from normal operation to leads-off state and vice versa.

In another embodiment of the invention the receiver unit is further arranged to comprise means for detecting radio wave disconnection state and/or means for signaling the radio wave disconnection state information to the patient monitor by feeding a predefined DC-voltage to the ECG signal port.

In yet another embodiment of the invention the output impedance of the receiver unit is less than 100E6 ohms. The output voltage of the receiver unit in leads-off state can further be greater than 1V.

The medical telemetry system according to the invention can further comprise an indicator for indicating the leads-off state to a user of the system. The receiver unit of the medical telemetry system according to the invention can further be arranged to comprise an indicator such as a LED for indicating the radio wave disconnection state to a user of the medical telemetry system.

In addition, the leads-off information and radio wave disconnection information can both independently activate the feeding of the DC-voltage to the ECG signal port.

In one embodiment of the invention, the transmitter unit comprises an impedance measurement circuit for detecting the leads-off state.

The transmitter unit and the receiver unit can further be paired so as to be interchangeably connected to each other.

The transmitter unit of the present invention can further comprise means for detecting a pacemaker in the said ECG signal. In order to transmit the pacemaker peak timing information to the monitor, sharp voltage spices are superimposed on the reconstructed ECG signal.

Benefits of the invention are related to the improved reliability of leads-off measurement and signaling in a medical telemetry system. Voltage mode signaling is more reliable than high impedance signaling. In addition, feeding a predefined low-impedance DC-voltage to the ECG signal port requires only relatively simple electronic circuitry and is therefore, also relatively inexpensive to manufacture. A further benefit of the invention is that it allows the medical professionals to move the patient safely and easily while providing the possibility to change a new monitor to receive the ECG and leads-off signals from the sensor unit. It also relieves the stress experienced by the patient and artifacts in the ECG signal caused by the stress because there is no need to disconnect and reapply the measuring electrodes to the chest of the patient as the sensor unit (the electrodes and the transmitter unit) of the present invention may stay in place even if the patient is moved from one location to another.

Various other features, objects, and advantages of the invention will further be apparent from the following detailed description and the drawings.

### BRIEF DESCRIPTION OF THE DRAWINGS

The accompanying drawings, which are included to provide a further understanding of the invention and constitute a part of this specification, illustrate embodiments of the invention and together with the description help to explain the principles of the invention. In the drawings:

FIG. 1 is schematic presentation of a simplified prior art leads-off detecting circuit;

FIG. 2 depicts a simplified electrical circuitry of a receiver unit of a medical telemetry system, in which the output impedance is changed in response to a detected leads-off state;

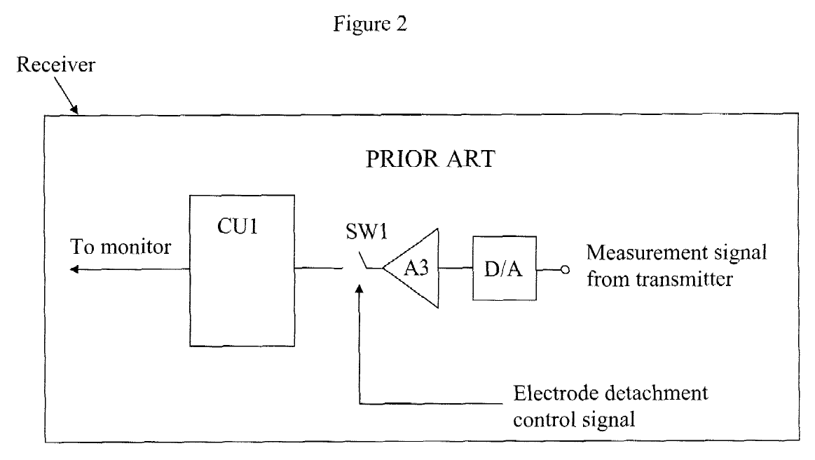

FIG. 3 depicts the ECG measurement system according to an embodiment of the present invention; and

FIG. 4 is a simplified presentation of the electronics circuits of the transmitter and receiver units according to an embodiment of the present invention.

### DETAILED DESCRIPTION OF THE EMBODIMENTS

Reference will now be made in detail to the embodiments of the present invention, examples of which are illustrated in the accompanying drawings.

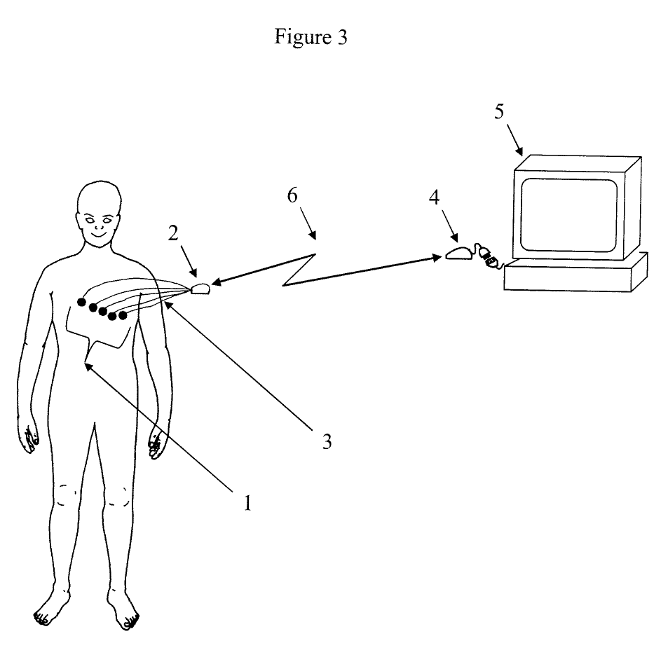

The ECG measurement system according to an embodiment of the present invention is illustrated in FIG. 3. The sensing unit comprises electrodes 1 connected to the patient for detecting biometric voltages and further connected to a transmitter unit 2 by leads 3 for transmitting the detected signals. Transmitter unit 2 contains e.g. a high gain amplifier, an analog to digital (A/D) converter and an antenna. For power acquisition, the transmitter may use a chargeable battery. The transmitter 2 performs the impedance measurement of the electrodes and detects when there is a poor electrode-to-skin connection or a lead detachment situation i.e. leads-off state of the electrodes occurs. When one or more of the electrodes is poorly attached on the skin, the transmitter unit 2 outputs a leads-off state signal and transmits it to the receiver unit using radio link 6. The transmitted signal is received by the receiver unit 4 and sent to the ECG signal port of the patient monitor and to ECG monitor 5 for further signal processing. The receiver unit 4 also contains a amplifier and an antenna. In addition, it contains a digital to analog (D/A) converter. The receiver unit may further comprise an indicator such as a LED for indicating the leads-off state to the user of the system. The ECG monitoring system also detects if the patient has a pacemaker. If a pacemaker is detected, time stamps are regularly added to the transmitted ECG signal and in the receiver unit a narrow peak is added to the signal, which looks like a pacemaker to the monitor.

When the receiver unit receives the leads-off state information, it signals the leads-off state to the monitor by driving a predefined low-impedance DC-voltage to the output pins of the receiver unit. The signaling unit signaling the leads-off state to the monitor thus comprises at least the DC-voltage source. This voltage is of the order of 2 V, whereas the ECG signal under normal conditions is significantly smaller (usually about 1-10 mV). When the leads-off state disappears, the DC voltage signal for the monitor is removed.

The transmitter and receiver units can be paired to operate together interchangeably. This can be implemented e.g. by push-buttons in the receiver/transmitter units that are pressed simultaneously to establish the initial set-up communication between the transmitter and the receiver units. Pressing buttons simultaneously may temporarily interrupt the measurement function in the transmitter unit. Initial set-up to accomplish pairing is done every time when a new patient is connected to the monitor. If the patient is transferred from one care area to another, it is convenient to keep the measurement electrodes at place and the sensor unit (the electrodes and the transmitter) active. When the patient enters a new care area with the sensor unit, there is another monitor with receiver unit waiting. After the set-up communication of the receiver and transmitter units, the receiver will accept only information from the corresponding transmitter and ignore other transmissions if there are other equipment functioning in the same frequency band.

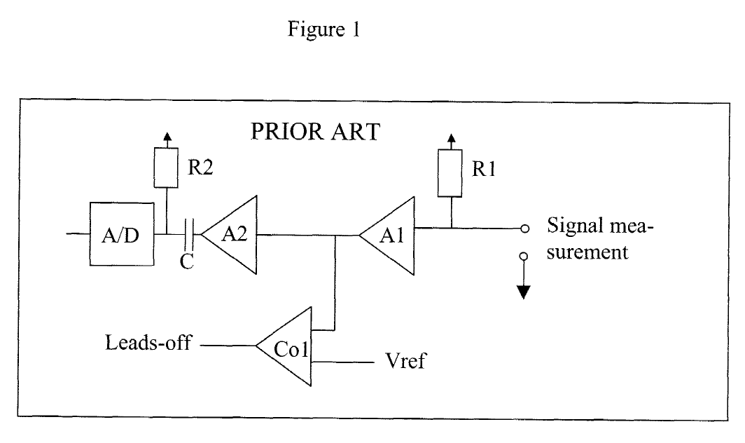

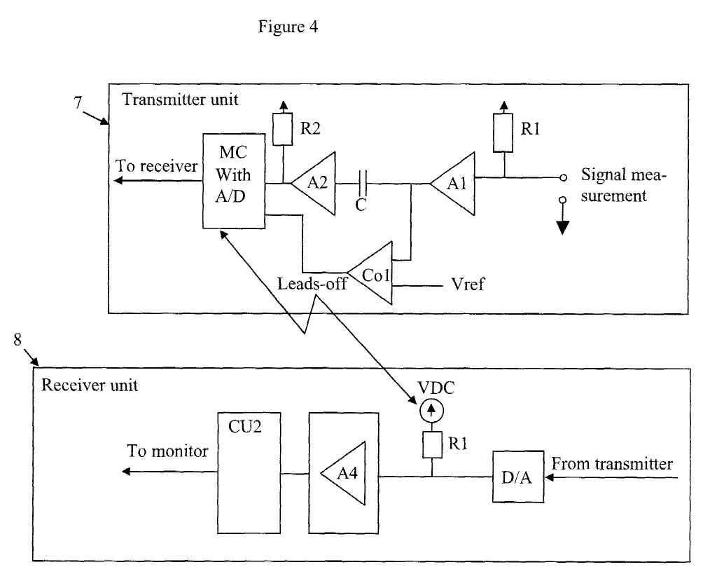

FIG. 4 illustrates the simplified structure of the transmitter and receiver units according to the present invention. The transmitter unit 7 performs the impedance measurement of the electrodes and in case of detecting a leads-off state, transmits leads-off information to the receiver unit 8. The impedance measurement is implemented in the transmitter similarly to prior art measurement described in the section referring to FIG. 1. The transmitter further includes a microcontroller MC with A/D converter and other circuitry needed for implementing digital transmission protocols such as the Bluetooth™. When the receiver 8 receives the leads-off signal from the transmitter, the voltage level at the input side of the amplifier unit A4 is altered by feeding a predefined low-impedance DC-voltage V_{DC} to the amplifier unit A4. The low-impedance output stage of the amplifier unit A4 is connected directly to the connector unit CU2. The “leads-off information” thus changes the output voltage of the D/A conversion unit, which output voltage is transferred by the amplifier unit A4 to connection unit CU2. The output impedance of the amplifier unit determines also the impedance seen from the connection unit. In the system according to the present invention, the output impedance of the amplifier unit A4 is constantly low regardless of the level of the output voltage. Because of the prior art design of the impedance measurement, the high DC voltage has same logical effect for the amplifier as high output impedance. This means that the monitor processes the high DC voltage in the amplifier output similarly to high output impedance of the amplifier unit A4. Generating a high DC voltage is technically advantageous over high output impedance, because it leads to less complex electronics circuit in the receiver unit.

In addition to a leads-off state, ECG signal acquisition can be disconnected if the radio wave communication between the transmission unit and the receiver unit is interrupted. If the receiver is unable to detect a signal a radio wave disconnection state is activated in the receiver unit. The receiver unit indicates the radio wave disconnection state for the user using a visual indicator (e.g. a LED) on the receiver unit. This indicator is different from the indicator for the leads-off state of the electrodes although there may also be another indicator for the leads-off state in the receiver. The receiver unit signals the radio wave disconnection state to the monitor by driving a predefined DC-voltage to its output pins. Therefore, irrespective of which state (radio wave disconnection state or leads-off state) receiver unit detects, its function is the same and a DC voltage is output from the receiver unit to the monitor. Consequently, on the monitor display “pleads-off” message is displayed for both disconnections and additional information about the state of the radio link and/or the leads-off state can be obtained from visual indicators on the receiver unit.

Inside the receiver unit, these two states are exclusive. Information on the leads-off state of the electrodes comes from the sensor unit via radiofrequency RF link. If the RF communication is interrupted, radio wave disconnection state is activated and leads-off state is deactivated, because no such information is available. To the monitor port these both states generate an identical message in the form of a high DC voltage, but when radio wave disconnection state is activated there is no more real-time or memory-stored information about the possible leads-off state in the receiver or in the monitor. Hence, in such a transition state, where leads-off state is active at the moment when radio wave disconnection state is activated, the high DC voltage is maintained without interruption. The user may first check and correct the radio link and then determine whether the leads-off state is still activated.

It must be contemplated that the above embodiments of the invention are presented here as examples and that the basic idea of the invention may vary within the scope of the claims. It will also be evident to a person skilled in the art that with the advancement of technology, the idea of the invention may be implemented in various other ways. The invention and its embodiments are thus not limited to the examples described above; instead they may vary within the scope of the claims.

What is claimed is:

1. A medical telemetry system, the system comprising:
a patient monitor comprising an ECG input connector and a signal port;
a sensor unit in proximity with the patient further comprising a transmitter unit and ECG electrodes to be attached on a patient and to be connected to the transmitter unit;
a receiver unit connected to ECG input connector on a patient monitor, and configured to receive ECG signal and leads-off state information via wireless radio-wave communication from the transmitter unit, and configured to feed a reconstructed ECG signal to the input connector of the patient monitor; and
a signaling unit for signaling leads-off information to the patient monitor by feeding a predefined DC-voltage to the ECG signal port.

2. The medical telemetry system according to claim 1, wherein the output impedance of the receiver unit is configured to remain constant when changing over from normal operation to leads-off state.

3. The medical telemetry system according to claim 1, wherein said receiver unit is further configured to detect radio wave disconnection state.

4. The medical telemetry system according to claim 3, wherein said receiver further comprises:
signaling unit for signaling radio wave disconnection state information to the patient monitor by feeding a predefined DC-voltage to the ECG signal port.

5. The medical telemetry system according to claim 1, wherein the output impedance of the receiver unit is less than 100E6 ohms.

6. The medical telemetry system according to claim 1, wherein the output voltage of the receiver unit in leads-off state is greater than 1 V.

7. The medical telemetry system according to claim 1, wherein said transmitter unit comprises an impedance measurement circuit configured to detect the leads-off state.

8. The medical telemetry system according to claim 1, wherein said system further comprises an indicator configured to indicate the leads-off state to a user of the system.

9. The medical telemetry system according to claim 3, wherein the receiver unit further comprises:
an indicator configured to indicate the radio wave disconnection state to a user of the medical telemetry system.

10. The medical telemetry system according to claim 1, wherein said transmitter unit and said receiver unit are arranged to be paired so as to be interchangeably connected to each other.

11. The medical telemetry system according to claim 1, wherein said transmitter unit is further configured to detect a pacemaker in the said ECG signal.

12. The medical telemetry system according to claim 11, wherein sharp voltage spikes are arranged to be superimposed on the reconstructed ECG signal in order to transmit a pacemaker peak timing information to the monitor.

13. The medical telemetry system according to claim 3, wherein at least one of the leads-off state information and the radio wave disconnection state information is arranged to activate the feeding of the DC-voltage to the ECG signal port.

14. A method for signaling leads-off information in a medical telemetry system, the method comprising:
arranging a sensor unit in proximity with a patient, the sensor unit comprising a transmitter unit and ECG electrodes to be attached on a patient and to be connected to the transmitter unit;
receiving by a receiver unit connected to ECG input connector on a patient monitor comprising an ECG signal port, ECG signal and leads-off information via wireless radio-wave communication;
feeding by said receiver unit a reconstructed ECG signal to an input connector of the patient monitor; and
signaling the leads-off information to the ECG monitor by feeding a predefined DC-voltage to the ECG signal port.

15. The method for signaling leads-off information in a medical telemetry system according to claim 14, wherein the method further comprises the step:
arranging the output impedance of the receiver unit to remain constant when changing over from normal operation to leads-off state.

16. The method for signaling leads-off information in a medical telemetry system according to claim 14, wherein the method further comprises the step:
detecting a radio wave disconnection state by said receiver unit.

17. The method according to claim 16, wherein the method further comprises the step:
signaling said radio wave disconnection state information to the patient monitor by feeding a predefined DC-voltage to the ECG signal port.

18. The method according to claim 14, wherein the method further comprises:
adjusting the output impedance of the receiver unit to less than 100E6 ohms.

19. The method according to claim 14, wherein the method further comprises:
adjusting the output voltage of the receiver unit in leads-off state to greater than 1 V.

20. The method according to claim 14, wherein the method further comprises:
detecting the leads-off state by an impedance measurement circuit in said transmitter unit.

21. The method according to claim 14, wherein the method further comprises:
indicating by an indicator of said system the leads-off state to a user of the system.

22. The method according to claim 16, wherein the method further comprises:
indicating by an indicator of said receiver unit the radio wave disconnection state to a user of the medical telemetry system.

23. The method according to claim 14, wherein the method further comprises:
pairing said transmitter unit and said receiver unit so as to be interchangeably connected to each other.

24. The method according to claim 14, wherein the method further comprises:
detecting a pacemaker from the ECG signal by said transmitter unit.

25. The method according to claim 24, wherein the method further comprises:
superimposing sharp voltage spikes on the reconstructed ECG signal in order to transmit a pacemaker peak timing information to the monitor.

26. The method according to claim 16, wherein the method further comprises:
activating the feeding of the DC-voltage to the ECG signal port by at least one of the leads-off information and the radio wave disconnection information.
